(19) United States
(12) Patent Application Publication
(10) Pub. No.: US 2007/0276303 A1
(43) Pub. Date: Nov. 29, 2007

(54) Gripping Device and Method

(75) Inventor: Thomas W. Jenner JR., Seattle, WA (US)

Correspondence Address: SEED INTELLECTUAL PROPERTY LAW GROUP PLLC, 701 FIFTH AVE, SUITE 5400, SEATTLE, WA, 98104, US

(73) Assignee: NEW SCHOOL TECHNOLOGIES, LLC, MERCER ISLAND, WA (US)

(21) Appl. No.: 10/573,665

(22) PCT Filed: Oct. 7, 2004
(86) PCT No.: PCT/US04/32856

§ 371 Date: Jul. 3, 2007

Related U.S. Application Data
(60) Provisional application No. 60/509,452, filed on Oct. 8, 2003.

(51) Int. Cl. A61F 5/01

(52) U.S. Cl. 602/21

(57) ABSTRACT

A device designed to assist individuals who have limited use of their hands, the device including a motor-controlled clamp for gripping objects, a microprocessor coupled to the motor-controlled clamp for actuating the motor, an input transducer coupled to the controller to receive input commands from the user to initiate action by the motor-controlled clamp to selectively grip and release an object, and a base for removably attaching the device to the limb of a user, either via straps or to a cast or brace using fasteners.

### BACKGROUND OF THE INVENTION

1. Field of the Invention

The present invention pertains to a device and method for augmenting a functional limb and, more particularly, to a mechanism worn on or attached to a user's arm, wrist, or hand that responds to user commands via an input transducer to activate a motor-controlled clamp for selectively grasping, manipulating, and releasing objects.

2. Description of the Related Art

Individuals whose work or hobby calls for long periods of steady hand movement can suffer disabling injuries. Repetitive stress syndrome occurs to anyone—computer operators, photographers, graphics designers, and anyone whose work or hobby calls for long periods of steady hand movement, and repeated grasping, turning, and twisting.

Injury results when repeated motions case the finger tendons to swell inside a narrow passageway formed by the carpal bones and a tough layer of ligaments. The meridian nerve passes through this tunnel and controls sensation in the fingers, thumb, and some muscles in the hand. Swelling of the tendons causes them to press the nerve against the bone. If the movement patterns that cause the symptoms continue, the hands can eventually lose their ability to firmly grasp objects or even suffer permanent damage.

Numerous mechanical and electro-mechanical devices have been developed over the years to aid in reducing stress or providing disabled individuals the means for picking up, manipulating, and releasing objects. Although these devices more or less function to meet their objectives, they tend to be cumbersome, clumsy, complicated, and expensive to manufacture and maintain. Examples of such devices are described below.

A device for controlling and operating paralyzed hands is disclosed in U.S. Pat. No. 2,553,277 wherein multiple springs and cables are connected to the hand, wrist, and finger shields worn by a user. The device is configured so that articles are picked up, gripped, and held between the fingers and the thumb of the user using a mechanical linkage system coupled between the wrist shield and hand and thumb shields that are all eventually coupled to a harness worn over the limb or muscular structure of a user. Thus, this device requires almost marionette-like movement by the user's body to effectuate movement of the paralyzed hand and fingers.

A power-assisted upper extremity orthosis is disclosed in U.S. Pat. No. 5,800,561 in which a device controlled by a trigger mechanism operated by one or more fingers of a user operates a pressurized gas supply and electric battery power to operate a mechanical gripping member that is designed to relieve pressure on a users joints suffering from an arthritic condition. This device requires not only an electric motor and power source but also a pneumatic cylinder and gas-operated piston.

In U.S. Pat. No. 3,631,542, a myoelectric brace consisting of a first wrist-hand splint portion having a movable finger support portion pivotally secured thereto is operated by a hydraulic actuator. The actuator is coupled to a pump that is driven by a battery-powered direct current motor. Three skin electrodes are positioned on the patient's arm and are configured to sense muscle potentials in the patient's arm when the patient tenses a muscle in the immediate area of the skin electrodes. The resulting myo-potentials are then amplified by a muscle potential amplifier that transforms the potentials into a varying control signal by a detector circuit and a filter circuit. Relaxation of the patient's muscle causes the finger support to pivotally move away from the fixed splint portion. This device requires hoses, a fluid chamber, and a fluid-operated actuator in combination with electric circuits in order to operate.

### BRIEF SUMMARY OF THE INVENTION

The disclosed embodiments of the invention are directed to a device designed to assist people who either must engage in repeated hand movement or who have limited use of their limbs, and in particular their hands, for providing a mechanism to grasp, pick up, manipulate, utilize, and release objects. An intuitive control switch is coupled with a motor-controlled clamp. In most applications, the motor can comprise a commercially-available servo used by hobbyists for radio-control model aircraft, boats, and cars.

In one embodiment, the device is capable of opening and closing in a precise motion that is suitable for assisting in everyday tasks and is useful in physical rehabilitation as well as in construction or assembly environments where constant grasping may prove hazardous to worker health.

In accordance with another embodiment, a device for aiding a user in gripping objects is provided that includes gripping members configured for gripping objects that include at least one movable gripping member, an actuator coupled to the movable gripping member to effectuate movement of the movable gripping member; a controller coupled to the actuator to control actuation of the actuator; an input transducer coupled to the controller and configured to receive input commands to cause the controller to initiate action by the actuator to move the movable gripping member and selectively grip and release an object; and a mounting device for removably mounting the gripping member, actuator, controller, and input transducer to the user.

In accordance with another aspect of the foregoing embodiment of the invention, the actuator preferably comprises a servo of the radio-controlled model craft type or a DC gear head motor configured to produce from 20 inch-ounces of torque to 600 inch-ounces of torque. The servo is preferably linked with straight linkage between the mounting member and the movable gripping member.

In accordance with another aspect of the foregoing embodiment, the controller comprises a microcontroller that, when not in use, operates in a sleep mode to conserve power, and when activated by movement of the user's body, such as pronation or supination of the hand or flexing of the hand about the wrist, initiates activation of the servo.

### BRIEF DESCRIPTION OF THE DRAWINGS

FIG. 1 is a block diagram of one embodiment of a gripping device formed in accordance with the present invention;

FIG. 2 is an isometric view of a representative implementation of the gripping device of the present invention;

FIG. 3 is an isometric view of an alternative embodiment of a gripping device formed in accordance with the present invention;

FIG. 4 is an isometric view of an alternative embodiment of a gripping device formed in accordance with the present invention;

FIG. 5 is an isometric view of an actuation mechanism of the embodiment of FIG. 4; and

FIG. 6 is an isometric view of a further embodiment of the gripper device formed in accordance with the present invention.

### DETAILED DESCRIPTION OF THE INVENTION

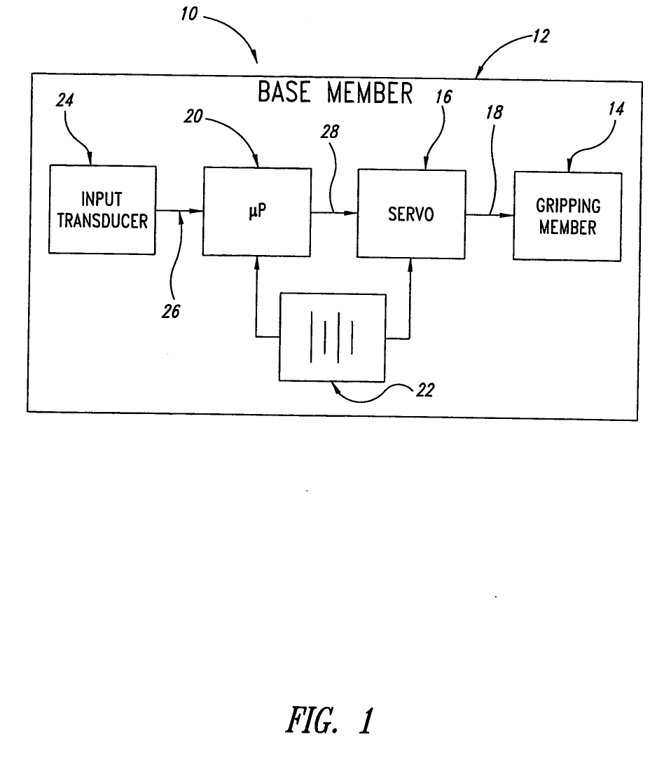

Referring initially to FIG. 1, shown therein is a block diagram illustrating the basic components of a gripping device 10 formed in accordance with one embodiment of the present invention. The gripping device 10 includes a base member 12 upon which the other components of the gripping device 10 are mounted and which is used for attaching the gripping device 10 to the limb of a user, such as an arm, wrist, or hand, or a combination thereof, or to a foot, leg, or other part of the body.

Mounted on the base member 12 is a gripping mechanism 14 composed of at least one movable gripping member. Ideally one fixed gripping member is provided, although it is not required. The at least one movable gripping member is pivotally mounted to the base member 12. Alternatively, two movable gripping members 14 that cooperate to hold and release objects can be pivotally mounted to the base member 12. Optional attachments for the gripping members 14 can be provided to accommodate different sizes and shapes of objects. Interchangeable gripping members can be used as well.

The gripping members 14 can comprise multiple gripping members pivotally mounted to the base member 12 to cooperate with a fixed gripping member (not shown) or with each other. Alternatively, flexible gripping members 14 can be used without a hinge to save weight, although such flexible material must be of the type that can handle repeated bending without fatigue or failure.

The gripping members 14 are coupled to a servo 16 via a linkage 18. The servo 16 is configured to cause movement of the movable gripping member or members 14 to grasp, manipulate, and release objects. The servo 16 is controlled by a controller 20, preferably a microcontroller, that is electrically coupled thereto. Both the microprocessor 20 and the servo 16 are energized by a power source 22, preferably a rechargeable battery, such as a lithium battery. Coupled to an input of the microprocessor 20 is an input transducer 24 that receives commands from a user and in response thereto generates command signals 26 that are received by the microprocessor 20. The microprocessor 20 in turn generates control signals 28 to the servo 16.

In a preferred embodiment, the servo 16 comprises a servo of the type used in radio-controlled model airplanes and cars. Such servos are ideal for this type of application because of their light weight and low power requirements, yet they have sufficient torque to provide the necessary force to grasp objects. Alternatively, a DC gear head motor configured to provide 20 inch-ounces of torque to 600 inch-ounces of torque can be used. While hydraulic or pneumatic actuated servos can be used, such are not preferred because of the required additional hardware that adds weight and complexity.

In a simple version, the gripping members 14 operate between an opened configuration and a closed configuration in response to a signal from the microprocessor 20 through the servo 16. In one instance the signal can be a binary on-off signal. However, additional features can be added, including proportional movement in response to the strength of the input signal 26 received from the input transducer 24, a stepped response to the input signal 26, or a ratchet-type of response where the gripping members 14 are configured to close in steps and to open in steps. A port on the microprocessor 20 can be included to allow modification of the microprocessor's reaction to the input signal 26, such as through software modifications downloaded to a register or memory associated with the microprocessor directly or via wireless transmission.

The microprocessor 20 is of conventional construction and readily commercially available, and hence it will not be described in detail herein. Preferably, the microcontroller has a sleep mode that, when not actively used, conserves power yet remains available upon receipt of a wake-up signal from the input transducer 24.

The input transducer 24 preferably comprises a resistor coupled to the base member in such a way that it changes its resistance in response to movement of the user's arm, wrist, hand, or a combination thereof. One type of resistor is a bendable resistor that is commercially available. Alternatively, the transducer can comprise other input devices that send a signal, preferably a binary signal, in response to user input. For example, the input transducer 24 can be configured as an eye mouse to detect eye movement. Another form of input is the detection of brainwaves, such as alpha, beta, or theta waves, detected from sensors attached to the user's head. However, such sensors require additional wiring, and hence this is not a preferred embodiment of the invention. To maintain simplicity and avoiding the use of electrodes attached to the user's skin, the bendable resistor is an ideal component.

For general purpose use, the base member 12 is preferably formed of lightweight plastic with bendable resistors associated therewith. The resistor can be embedded within the plastic or mounted thereon. Additional weight can be saved by using plastic gears in the servo 16. However, for heavy duty applications, such as riding bicycles and scooters, the servo 16 and gripping members 14 can be strengthened through the use of metal gears and components. In addition, the base member 12 can be formed of rigid metal material, wood, composites, plastics, and the like. In this embodiment, the input transducer 24 could be a switch, or other input device that does not require flexing or bending of the base member 12.

For heavy duty use, the interchangeable gripping members can be adapted for specialized tasks. For example, a gripping member can be configured to grip the handle of cups, jugs, and the like. Another gripping member can be structured to facilitate grasping of elongate objects, such as knives, forks, and the like.

Preferably, the base member 12 is configured for mounting on a user's limb. However, it can also be mounted on a cast or brace worn by the user and affixed in place by a Velcro strap or threaded to studs or other mounting hardware in the cast or brace, or it can be integrally formed with the cast.

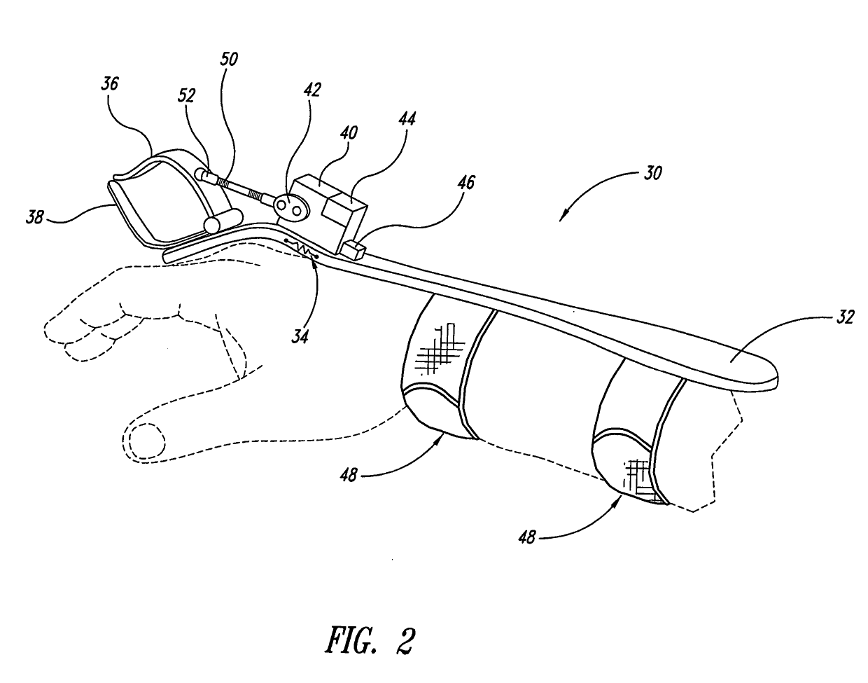

Referring next to FIG. 2, shown therein is an isometric view of a hand gripper 30 that includes a plastic base 32 having a bendable resistor 34, preferably integrally formed therewith. Extending from the base 32 is a pivotally-mounted grip 36 configured to cooperate with a fixed grip 38 also extending from and mounted to the base 32.

The movable grip 36 is coupled to a servo 40 via a linkage 42, which linkage 42 is adjustable to permit adjustment in the starting and ending position of the movable grip 36. The servo 40 is electrically coupled to a microcontroller 44 that in turn is electrically coupled to the bendable resistor 34 and a power source 46, in this case a battery. Velcro straps 48 attach the plastic base 32 to the limb of a user.

In use, the hand gripper 30 functions to augment the user's fingers and hand in grasping and picking up objects. When the user bends the plastic base 32, such as by flexing muscles in the wrist or forming a fist, the bendable resistor 34 changes its resistance. The bendable resistor 34 is formed as part of a voltage loop, causing a change in voltage at the microcontroller 44, which sends a wake-up signal to the microcontroller 44, which in turn initiates actuation of the servo 40 and causes movement of the movable grip 36. In one embodiment, the movable grip 36 moves towards the fixed grip 38 until the object is grasped between the movable and fixed grips 36, 38. The movable and fixed grips 36, 38 maintain tension on the object until the servo 40 moves the grip 36 in an opposite direction.

Movement of the grip 36 in a direction away from the object to thereby release the grasp can be initiated by the user relaxing the flexation of the muscles, which in turn changes the resistance of the bendable resistor 34, thereby altering the voltage received at the input to the microcontroller 44. In another embodiment, the microcontroller 44 can be configured to operate as a toggle switch in combination with the bendable resistor whereby relaxation of the user's muscles results in the movable and fixed grips 36, 38 maintaining their tension. The tension is released only when the user flexes their muscles a second time, in which case the microcontroller actuates the servo 40 to operate in a reverse direction, causing the movable grip 36 to move away from the fixed grip 38 and release the object.

As discussed above, the microcontroller 44 can be configured to operate the movable grip 36 in a stepped manner so that it moves a measured distance each time the user causes the resistor 34 to bend. Alternatively, the microcontroller 44 in combination with the servo 40 can operate in a proportional manner, i.e., pivoting the movable grip 36 a distance corresponding to the amount of muscle flexion or corresponding to the length of time the muscle is flexed. Hence, variations in the manner of operation can be programmed into the microcontroller 44.

In the embodiment shown in FIG. 2, the linkage 42 coupling the servo 16 to the grip 36 is preferably coupled in a straight line to resist backing out due to holding torque. However, the linkage 42 can be adjustable, and a locking mechanism can also be provided to maintain the gripping members 14 in a preferred condition. The adjustable linkage is used to enable positioning of the movable grip 36 at ending and starting positions. In this illustration, the linkage 42 includes a threaded rod 50 received into a rod end 52 for adjustment in the position of the rod end 52 relative to the servo 40. Rotation of the rod 50 moves the rod end 52 further away from or closer to the servo 40, which adjusts the movable grip 36 a corresponding distance. It is to be understood that other forms of linkage may be used between the servo 16 and the grip 36 as known to those skilled in the art.

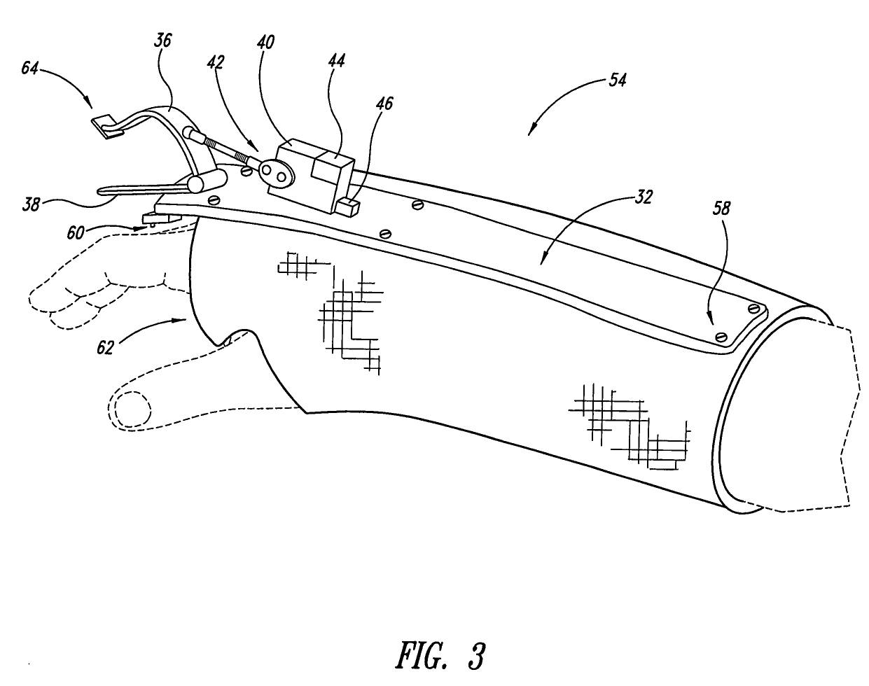

Referring next to FIG. 3, shown therein is another embodiment of the invention wherein a hand gripper 54 is shown attached to a cast 56 by a plurality of fasteners 58. In this embodiment, like elements from previous embodiments are referred to with identical reference numbers. Here, the base 32 is attached to a cast 56 to provide a more stable platform for heavy-duty applications. Ideally, the base 32 is formed of more rigid material, such as metal. Because the base 32 does not bend, a bendable resistor used in the previous embodiment is not suitable. Instead, a switch 60 is mounted at the proximal end 62 of the base 32 that can be tripped by flexion of the user's wrist, hand, or finger. Tripping of the switch 60 activates the microcontroller 44 mounted on the servo 40. It is to be understood that other methods of accepting user input can be used in this embodiment, as previously discussed.

The movable grip 36 includes a plate 64 mounted at the end thereof to aid in manipulating and holding objects having a more planar configuration. The movable grip 36 and the fixed grip 38 can each be configured to be interchangeable with other each other and with other gripping members that are designed to hold specific tools or handles.

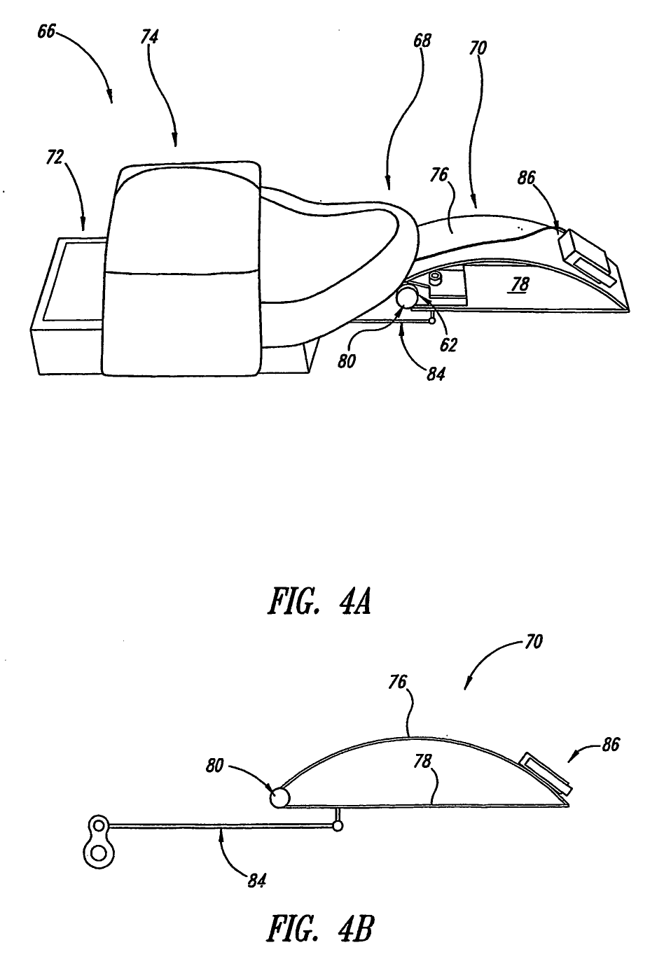

FIG. 4 is an isometric illustration of another embodiment of the invention in which a gripping device 66 is shown to include a base member 68 that includes a device (not shown) for sensing bending of the base member 68, a gripping mechanism 70 mounted on the base member 68, and an actuation system 72 coupled to the sensing device and to the gripping mechanism 70, the actuation system 72 configured to open and close the gripping mechanism 70 in response to bending of the base member 68. The device 66 further includes a releasable and adjustable strap 74 for attaching the device 66 to the body of a user, such as to the users arm or wrist or both.

The gripping mechanism 70 includes a curved fixed grip 76 and a substantially planar movable grip 78 pivotally attached to the fixed grip 76 by a hinge 80. A helical spring 82 formed as part of the hinge 80 biases the movable grip 78 into contact with the fixed grip 76. An adjustable link 84 is coupled to the movable grip 78 and to the actuation system 72 such that when the actuation system responds to the sensing device to open the gripping mechanism 70, the adjustable linkage 84 pulls the movable grip 78 away from contact with the fixed grip 76. The adjustable linkage 84 may be formed from string, cable, or a flexible filament that is lightweight yet strong enough for this application. Alternatively, a rigid rod that is adjustable in length or nonadjustable, as desired, may be used. The sensing device includes a switch 86, which is shown in FIG. 4 positioned on the outside surface of the fixed grip 76. The switch is configured such that when it is open, the movable grip 78 is urged into contact with the fixed grip 76 by the helical spring 82. When the switch is closed, the actuation mechanism 72 pulls the fixed grip through the adjustable linkage 84 into an open position by rotating it about the hinge 80 out of contact with the fixed grip 76.

The actuation mechanism 76 can be configured to move the movable grip 78 between an open position and a closed position in response to a digital signal, such as a binary signal. Alternatively, the actuation mechanism 72 can be configured to move the movable grip 78 in steps away from the fixed grip 76 and allow it to be urged towards the fixed grip 76 in steps, or to allow it to close immediately from any stepped open position. In accordance with another embodiment, the actuation mechanism 72 can be configured to provide proportional movement of the movable grip 78 in response to proportional bending of the base member 68. In this embodiment, the bendable resistor described above with respect to FIG. 1 could be used.

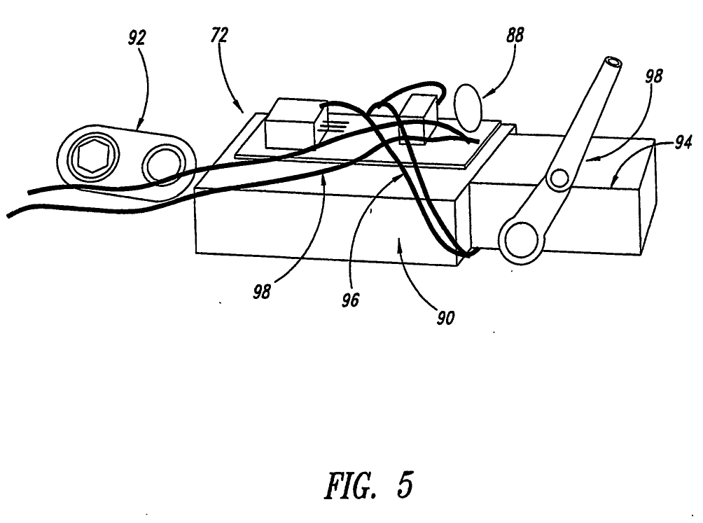

FIG. 5 is a detailed illustration of the actuation mechanism 72. As shown therein, the actuation mechanism includes a microprocessor 88 coupled to a power source 90, in this case a nine-volt battery 90. The battery 90 is coupled to the microprocessor 88 via a standard snap-on connector 92. A servo motor 94 is wired to the microprocessor 88 via a first pair of conductors 96. A second pair of conductors 98 couples the microprocessor 72 to the sensing device (not shown). An actuator arm 98 is coupled to the servo motor 94 and configured for attachment to the adjustable linkage 84 of FIG. 4.

It is to be understood that the actuation mechanism may be formed of integrated circuits, discreet components, or a combination of the same. In addition, lightweight components for the servo motor 94 and the battery 90 may be used to reduce the weight and size of the gripper device.

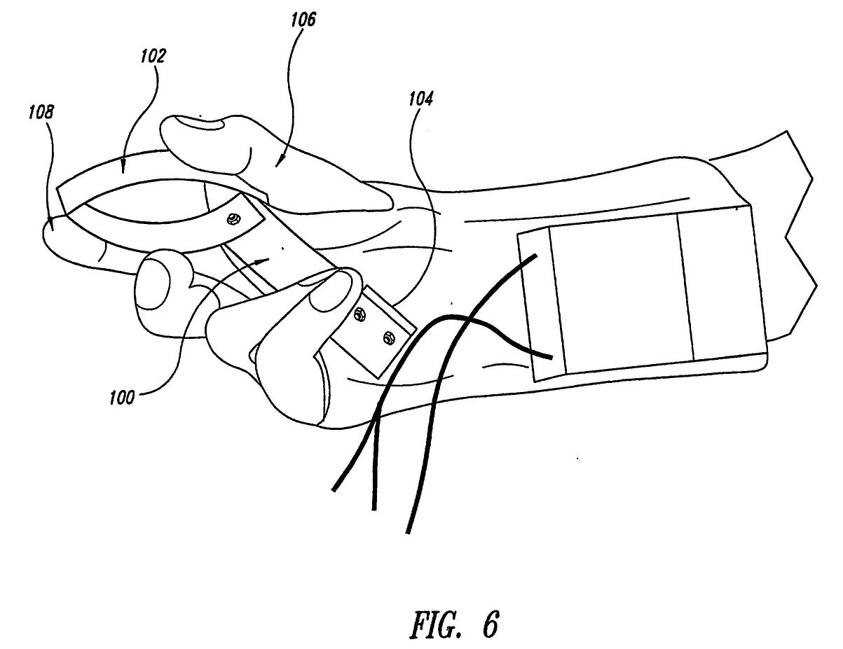

In order to provide a more natural feel, an alternative embodiment of the invention shown in FIG. 6 includes an offset plate 100 that couples the gripping mechanism 102 to the base member 104. The offset plate 100 positions the gripping mechanism 102 between the thumb 106 and the first finger 108 of the user. It is to be understood that straps or other means for retaining the thumb and finger 106, 108 in contact with the gripping mechanism 102 may be used as needed.

From the foregoing it will be appreciated that, although specific embodiments of the invention have been described herein for purposes of illustration, various modifications may be made without deviating from the spirit and scope of the invention. Accordingly, the invention is not limited except as by the appended claims and the equivalents thereof.

1. A device for aiding users in gripping objects, comprising:
means for gripping objects, including at least one movable gripping member;
means for moving the at least one movable gripping member to effectuate gripping and releasing of objects;
means for controlling the moving means;
transducer means coupled to the controlling means and configured to receive input commands from the user to cause the controller means to initiate action by the moving means to move the at least one movable member and selectively grip and release an object; and
means for removably mounting the device on a user.

2. A device for gripping, comprising:
a bendable base member;
a device for sensing bending of the base member;
a gripping mechanism mounted on the base member; and
an actuation system coupled to the gripping mechanism and to the sensing device, the actuation system configured to open and close the gripping mechanism in response to bending of the base member.

3. The gripping device of claim 2 wherein the base member is configured to be mounted on a user's body.

4. The gripping device of claim 2 wherein the sensing device is integrally formed with the bendable base member.

5. The gripping device of claim 4 wherein the sensing device comprises a bendable resistor.

6. The gripping device of claim 2 wherein the gripping mechanism comprises a movable gripping member coupled to the actuation system.

7. The gripping device of claim 6, further comprising a fixed gripping member configured to cooperate with the movable, gripping member for gripping objects.

8. The gripping device of claim 2 wherein the gripping mechanism comprises two movable gripping members configured to cooperate in gripping and releasing objects.

9. The gripping device of claim 2 wherein the gripping mechanism is configured to maintain tension in the closed position.

10. The gripping device of claim 2 wherein the actuation system comprises a microprocessor coupled to an actuator.

11. The gripping device of claim 10 wherein the actuator comprises a servo motor coupled to the microprocessor and linkage coupling the servo motor to the gripping mechanism.

12. The gripping device of claim 11 wherein the linkage comprises a spring configured to urge the gripping mechanism to close.

13. The gripping device of claim 12 wherein the linkage comprises an adjustable link.

14. The gripping device of claim 12 wherein the linkage comprises a flexible link coupling the servo motor to a movable gripping member.

15. The gripping device of claim 12 wherein the linkage comprises a flexible filament coupling the servo motor to a movable gripping member.

16. The gripping device of claim 11 wherein the actuator is configured to move the gripping mechanism between an open position and a closed position in response to a binary signal.

17. The gripping device of claim 11 wherein the actuator is configured to move a movable gripping member of the gripping mechanism in stepped fashion.

18. The gripping device of claim 11 wherein the actuator is configured to proportionally move at least one movable gripping member in the gripping mechanism when opening and closing the gripping mechanism in response to bending of the base member.
